(19) United States
(12) Patent Application Publication
(10) Pub. No.: US 2018/0169100 A1
(43) Pub. Date: Jun. 21, 2018

(54) Cancer Stem Cell Targeting Compounds

(71) Applicant: Godavari Biorefineries Limited, Mumbai (IN)

(72) Inventors: Sangeeta Srivastava, Mumbai (IN); Maithili Athavale, Mumbai (IN); Kedar Shukre, Mumbai (IN); Gayatri More, Mumbai (IN)

(21) Appl. No.: 15/125,323

(22) PCT Filed: Mar. 11, 2015
(86) PCT No.: PCT/IN2015/050020

§ 371 Date: Sep. 12, 2016

(30) Foreign Application Priority Data
Mar. 11, 2014 (IN) 815/MUM/2014

(51) Int. Cl. A61K 31/519; A61P 35/00

(52) U.S. Cl. CPC A61K 31/519; A61K 2300/00; A61P 35/00

(57) ABSTRACT

The present invention provides compounds of formula (I), compositions, uses thereof and methods for inhibiting proliferation or obliterating cancer stem cells which includes killing; and/or inducing apoptosis in cancer stem cells. Included within the scope of such compounds, compositions, uses thereof and methods are those in which proliferation of cancer stem cells are selectively eradicated or inhibited.

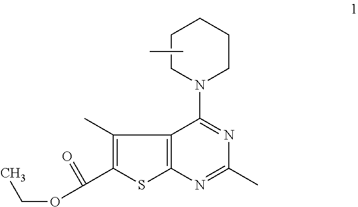

### FIELD OF THE INVENTION

The present invention relates to compounds for targeting cancer stem cells, compositions and uses thereof in arresting or inhibiting proliferation or obliterating cancer stem cells. The present invention also relates to a method of arresting or inhibiting proliferation or obliteration of cancer stem cells.

### BACKGROUND OF THE INVENTION

Cancer is regarded as imminent “human disaster” by the WHO, as the cancer cases are expected to surge 57% worldwide in the next 20 years. The incidence of cancer globally increased in just four years from 12.7 m in 2008 to 14.1 m new cases in 2012, when there were 8.2 m deaths. The World Cancer Report, produced by the WHO's specialized cancer agency, predicts new cancer cases will rise from an estimated 14 million annually in 2012 to 22 million within two decades. Over the same period, cancer deaths are predicted to rise from 8.2 million a year to 13 million. However, conventional as well as current approaches to treat cancer have not changed drastically and have not led to the improvement in survival rate. Such limitation in addition to other factors is considered to be due to the approach of essentially targeting cells which are fast-growing and form the bulk of the tumor. Such approaches do not target cancer stem cells which are regarded as an underlying cause of giving rise to tumor or cancer cells. Besides, with the use of chemotherapeutic agents, radiations and other regimens cancer stem cells are believed to survive and give rise to ‘second-line’ tumours with acquired resistance to the ‘first-line’ treatment. ‘Second-line’ tumours with higher resistance to therapy make them very hard to eliminate complicating further therapy.

These approaches thus have short comings of not only lack of effectiveness due to inability to target cancer stem cells including leukemia stem cells, but also suffer from drawbacks of resistance to conventional chemotherapeutic therapies as well as newer targeted therapies, and recurrence or relapse of cancer in patients. Therefore, there is unmet need of providing compounds or regimens which are able to target cancer stem cells.

### SUMMARY

In one embodiment, the present invention provides compounds having the general formula I or pharmaceutically acceptable derivatives thereof.

In certain embodiments, the present invention provides compounds having the formula I or pharmaceutically acceptable derivatives thereof including salts, solvtes, or hydrates for arresting or inhibiting proliferation or obliterating cancer stem cells, wherein:

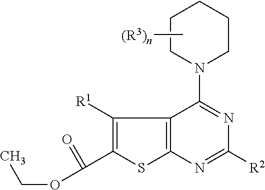

each R¹, R² and R³ is independently selected from halogen, C1-6haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

Each n is independently 0-5. In certain embodiments, n is 1-4. In some embodiments, n is 1-3. In yet other embodiments n is 1-2. In some embodiments, n is 0, 1, 2, 3, 4 or 5.

Compounds of the present invention include those described generally above, and are further illustrated by the classes, subclasses, and species disclosed herein. Various terms and terminology used hereinabove in describing the compounds of the present invention and all technical and scientific terms used herein have the same would mean or refer to standard definition or meaning or as used in a chemical or technical field or as known or commonly understood by one of ordinary skill in the art to which this invention belongs.

Compounds of the present invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

In certain embodiments, one or more substituent is individually and independently selected from alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

In certain embodiments, the present invention provides a pharmaceutically acceptable derivative of a compound of the formula II.

In one embodiment, the present invention provides compound of formula I or pharmaceutically acceptable derivative of compound of formula I or II including salts, solvtes, or hydrates for arresting or inhibiting proliferation or obliterating cancer stem cells:

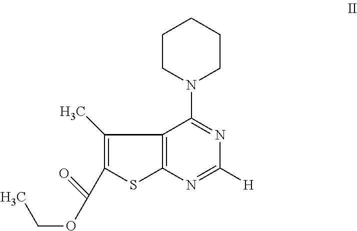 [SMILES: CCOC(=O)c1sc2ncnc(N3CCCCC3)c2c1C]

In certain embodiments, the present invention provides compositions comprising a therapeutically effective amount of the compound of formula I, or a pharmaceutically acceptable salt or derivative thereof and a pharmaceutically acceptable excipient including carrier, adjuvant, vehicle or mixtures thereof.

In certain embodiments, the present invention provides a composition for arresting or inhibiting proliferation or obliterating cancer stem cells comprising a compound having formula II or a pharmaceutically acceptable salt or derivative thereof and a pharmaceutically acceptable excipient including carrier, adjuvant, or vehicle. The preferable derivative may be a pharmaceutically acceptable ester, or salt of an ester.

The amount of compound in compositions of this invention may be such that it is effective in arresting or inhibiting proliferation or obliterating cancer stem cells, in a biological sample or in a subject in the need thereof. In certain embodiments, the amount of compound in compositions may be such that it is effective to measurably arresting or inhibiting proliferation or obliterating cancer stem cells, in a biological sample or in a subject in the need thereof. In certain embodiments, the composition may comprise between the biologically effective dose and the maximum tolerated dose of the compound of the invention or it's pharmaceutically acceptable salt, ester, or salt of an ester.

In certain embodiments, a composition of this invention may be formulated for administration to a subject in the need thereof.

Compositions of the present invention may be formulated into a suitable dosage form to be administered orally, parenterally, by inhalation spray, topically, rectally, nasally, buccally, vaginally or via an implanted reservoir. Compositions of the present invention may be formulated into dosage forms including liquid, solid, and semisolid dosage forms. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. Preferably, the compositions are administered orally, intravenously or intraperitoneally.

In one embodiment compounds having the general formula I or a pharmaceutically acceptable salt thereof or compositions thereof may be used for arresting or inhibiting proliferation or obliterating cancer stem cells and thereby treating associated disorders or diseases or conditions. Thus, provided compounds may be useful for treating cancers, including, but not limited to hematological cancers and solid tumors.

In certain embodiments compounds of the present invention having the formula II or a pharmaceutically acceptable salt thereof or compositions thereof may be used for arresting or inhibiting proliferation or obliterating cancer stem cells and thereby treating associated disorders or diseases or conditions. Thus, provided compounds are useful for treating cancers, including, but not limited to hematological cancers and solid tumors.

In another embodiment the present invention provides a method of arresting or inhibiting proliferation or obliterating cancer stem cells by administering compounds having the general formula I or a pharmaceutically acceptable salt or derivative thereof or compositions comprising the same in subjects in the need thereof.

In certain embodiments the present invention provides a method of arresting or inhibiting proliferation or obliterating cancer stem cells by administering the compound having formula II or a pharmaceutically acceptable salt thereof or compositions comprising the same in subjects in the need thereof.

According to one embodiment, the invention relates to a method of arresting or inhibiting proliferation or obliterating cancer stem cells in a biological sample comprising the step of contacting said biological sample with a compound of formula I or II or derivative thereof or composition comprising the same in an effective amount. In certain embodiments, the invention relates to a method of killing cancer cells or cancer stem cells in a biological sample comprising the step of contacting said biological sample with a compound of formula I or II or derivative thereof or composition comprising the same in an effective amount.

In one more embodiment the present invention provides a method of treatment of disorders or diseases or conditions associated with cancer stem cells by administering compounds of formula I or II or derivative thereof or compositions comprising the same in subjects in the need thereof.

In certain embodiments, the present invention provides a method for treating a disorder mediated by cancer stem cells in a patient in need thereof, comprising the step of administering to said patient a compounds of formula I or II or derivative thereof or composition comprising the same in an effective amount. Such disorders include cancer or recurrence or relapse of cancer or other proliferative diseases.

In certain embodiments, the invention relates to a method of eradicating arresting or inhibiting proliferation or obliterating cancer stem cells in a patient, leading to remission of the cancer, comprising the step of administering to said patient a compound of formula I or II or derivative thereof or composition comprising the same in an effective amount.

In some embodiments the compounds of formula I or II or derivative thereof or composition comprising the same in an effective amount may be used in a method of treating a cancer or other proliferative disorder. In some embodiments the present invention provides a method of treating a cancer or other proliferative disorder, comprising administering a compound or composition of the present invention to a patient with a cancer or other proliferative disorder.

In certain embodiments the compounds of formula I or II or derivative thereof or composition comprising the same in an effective amount may be used to treat a cancer in a human patient, said cancer occurring in the patient's prostate, breast, neck, colon, skin, liver, stomach, pancreas, kidney, ovary, lung, testicle, penis, thyroid, parathyroid, pituitary, thymus, retina, uvea, conjunctiva, spleen, head, trachea, gall bladder, rectum, salivary gland, adrenal gland, throat, esophagus, lymph nodes, sweat glands, sebaceous glands, muscle, heart, brain, blood, or bone marrow.

Depending upon the particular condition, or disease, to be treated, additional therapeutic agents, which are normally administered to treat that condition, may be administered in combination with compounds and compositions of this invention. In some embodiments, a provided compound of this invention, or composition thereof, is administered in combination with one or more other chemotherapeutic agents.

### BRIEF DESCRIPTION OF THE DRAWINGS

FIG. 1 is graph showing that the compound Ethyl-5,6-dimethyl-4-(piperidin-1-yl)thieno[2,3-d]pyrimidine has better anticancer effect on primary spheres of PC3 as compared to cisplatin.

FIG. 2 is graph showing that the compound Ethyl-5,6-dimethyl-4-(piperidin-1-yl)thieno[2,3-d]pyrimidine has better anticancer effect on primary spheres of DU145 compared to Cisplatin.

FIG. 3 is a graph showing that the compound Ethyl-5,6-dimethyl-4-(piperidin-1-yl)thieno[2,3-d]pyrimidine has better anticancer effect on primary spheres of MDA MB compared to Cisplatin.

FIG. 4 is quadrant plot of FACS results for PC3 cells without drug treatment, stained with anti-CD44-PE labeled and anti-CD24-FITC labeled antibodies showing CD44 expression in the 85.61% cells (UL) indicating a cell population rich with CSC or cancer stem like cells.

FIG. 5 is a quadrant plot of FACS Results for PC3 cells exposed to IC25 drug conc. of Cisplatin for 48 hrs showing exposure of Cisplatin IC25 drug conc. did not have much effect on CD44 population of PC3 cells indicating that it is not very effective on cancer stem cells.

FIG. 6 is a quadrant plot of FACS Results for PC3 cells exposed to IC25 drug conc. of Ethyl-5,6-dimethyl-4-(piperidin-1-yl)thieno[2,3-d]pyrimidine for 48 hrs: PC3 cells exposed to IC25 drug conc. of Ethyl-5,6-dimethyl-4-(piperidin-1-yl)thieno[2,3-d]pyrimidine showed a drastic effect on CD44 population that is almost a complete shift from CD44 region to the co-expressed region indicating the action of Ethyl-5,6-dimethyl-4-(piperidin-1-yl)thieno[2,3-d]pyrimidine on cancer stem cells of PC3 cells.

FIG. 7 is a bar graph showing that Ethyl-5,6-dimethyl-4-(piperidin-1-yl)thieno[2,3-d]pyrimidine exhibits better activity on cancer stem cells of PC3 compared to standard therapeutic drug Cisplatin.

FIG. 8 is quadrant plot of FACS results for DU145 cells without drug treatment, stained with anti-CD44-PE labeled and anti-CD24-FITC labeled antibodies showing CD44 expression in the 70.18% cells (UL) in quadrant plot indicating a cell population rich in cancer stem cells.

FIG. 9 is a quadrant plot of FACS Results for DU145 cells exposed to IC25 drug conc. of Cisplatin for 48 hrs showing exposure of Cisplatin IC25 drug conc. did not have much effect on CD44 population of PC3 cells indicating that it is not very effective on cancer stem cells.

FIG. 10 is a quadrant plot of FACS Results for DU145 cells exposed to IC25 drug conc. of Ethyl-5,6-dimethyl-4-(piperidin-1-yl)thieno[2,3-d]pyrimidine for 48 hrs: DU145 cells exposed to IC25 drug conc. of Ethyl-5,6-dimethyl-4-(piperidin-1-yl)thieno[2,3-d]pyrimidine showed a drastic effect on CD44 population that is almost a complete shift from CD44 region to the co-expressed region indicating the action of Ethyl-5,6-dimethyl-4-(piperidin-1-yl)thieno[2,3-d]pyrimidine on cancer stem cells of DU145 cells.

FIG. 11 is a bar graph showing that Ethyl-5,6-dimethyl-4-(piperidin-1-yl)thieno[2,3-d]pyrimidine exhibits better activity on cancer stem cells of DU145 compared to standard therapeutic drug Cisplatin.

### DETAILED DESCRIPTION OF THE INVENTION

Provided herein are compounds, compositions, uses thereof and methods for arresting or inhibiting proliferation or obliterating cancer stem cells which includes killing; and/or inducing apoptosis in cancer stem cells. Included within the scope of such compounds, compositions, uses thereof and methods are those in which proliferation of cancer stem cells are selectively arrested or inhibited or obliterated which includes killing, and/or inducing apoptosis relative to normal stem cells or any other normal cells.

Cancer stem cells have been reported to constitute a small fraction of cancer cells in a tumor. Cancer stem cells, due to their slow growing nature and slow replication are considered to be the hardest cells to eradicate in a cancer. The residual cancer stem cells remaining after elimination of cancer and other cells can then replicate and give rise to fresh cancer cells. Following treatment, there may be a period of remission followed by a period of recurrence. By inhibiting or obliterating cancer stem cells, the possibility of a cancer from recurring can be prevented or reduced. Also, treatment with compounds such as of the present invention which selectively arrest or inhibit or obliterate cancer stem cells can reduce the likelihood of adaption (resistance).

In one embodiment, the present invention provides compounds having the general formula I or pharmaceutically acceptable derivatives thereof.

In certain embodiments, the present invention provides compounds having the formula I or pharmaceutically acceptable derivatives thereof including salts, solvtes, or hydrates for arresting or inhibiting proliferation or obliterating cancer stem cells, wherein:

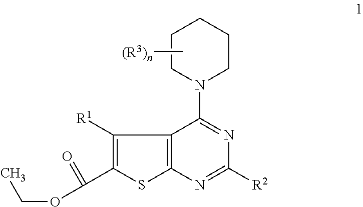

each R¹, R² and R³ is independently selected from halogen, C1-6haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

Each n is independently 0-5. In certain embodiments, n is 1-4. In some embodiments, n is 1-3. In yet other embodiments n is 1-2. In some embodiments, n is 0, 1, 2, 3, 4 or 5.

Compounds of the present invention include those described generally above, and are further illustrated by the classes, subclasses, and species disclosed herein. Various terms and terminology used hereinabove in describing the compounds of the present invention and all technical and scientific terms used herein have the same would mean or refer to standard definition or meaning or as used in a chemical or technical field or as known or commonly understood by one of ordinary skill in the art to which this invention belongs.

Compounds of the present invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

In certain embodiments, one or more substituent is individually and independently selected from alkyl, cycloalkyl, heteroalkyl, aryl, heteroaryl, heteroalicyclic, hydroxy, alkoxy, aryloxy, alkylthio, arylthio, alkylsulfoxide, arylsulfoxide, ester, alkylsulfone, arylsulfone, cyano, halo, alkoyl, alkoyloxo, isocyanato, thiocyanato, isothiocyanato, nitro, haloalkyl, haloalkoxy, fluoroalkyl, amino, alkyl-amino, dialkyl-amino, amido.

An “alkyl” group refers to an aliphatic hydrocarbon group. Reference to an alkyl group includes “saturated alkyl” and/or “unsaturated alkyl”. The alkyl group, whether saturated or unsaturated, includes branched, straight chain, or cyclic groups. By way of example only, alkyl includes methyl, ethyl, propyl, iso-propyl, n-butyl, iso-butyl, sec-butyl, t-butyl, pentyl, iso-pentyl, neo-pentyl, and hexyl. In some embodiments, alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. A “heteroalkyl” group substitutes any one of the carbons of the alkyl group with a heteroatom having the appropriate number of hydrogen atoms attached (e.g., a CH₂ group to an NH group or an O group).

An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=l, y=l and x=2, y=0. When x=2, the alkyl groups, taken together with the nitrogen to which they are attached, optionally form a cyclic ring system.

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(═O)OR, where R is selected from the group consisting of alkyl, cycloalkyl, aryl, heteroaryl and heteroalicyclic.

The term “carbocyclic” or “carbocycle” refers to a ring wherein each of the atoms forming the ring is a carbon atom. Carbocycles includes aryl and cycloalkyl groups. The term thus distinguishes carbocycle from heterocycle (“heterocyclic”) in which the ring backbone contains at least one atom which is different from carbon (i.e a heteroatom). Heterocycle includes heteroaryl and heterocycloalkyl. Carbocycles and heterocycles disclosed herein are optionally substituted.

As used herein, the term “aryl” refers to an aromatic ring wherein each of the atoms forming the ring is a carbon atom. Aryl rings disclosed herein include rings having five, six, seven, eight, nine, or more than nine carbon atoms. Aryl groups are optionally substituted. Examples of aryl groups include, but are not limited to phenyl, and naphthalenyl.

The term “cycloalkyl” refers to a monocyclic or polycyclic non-aromatic radical, wherein each of the atoms forming the ring (i.e. skeletal atoms) is a carbon atom. In various embodiments, cycloalkyls are saturated, or partially unsaturated. In some embodiments, cycloalkyls are fused with an aromatic ring. Cycloalkyl groups include groups having from 3 to 10 ring atoms. Illustrative examples of cycloalkyl groups include, but are not limited to, the following moieties:

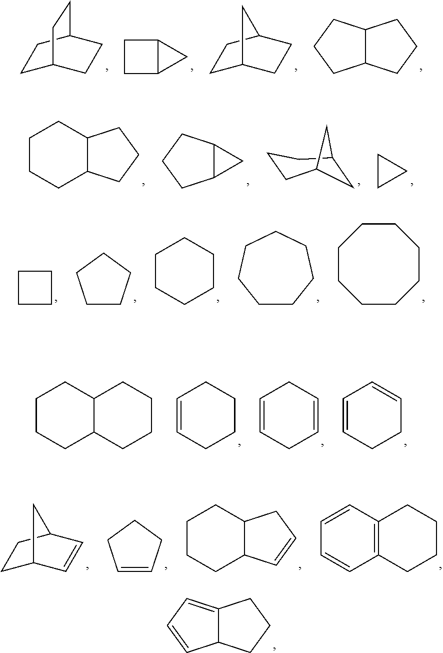

and the like. Monocyclic cycloalkyls include, but are not limited to, cyclopropyl, cyclobutyl, cyclopentyl, cyclohexyl, cycloheptyl, and cyclooctyl.

The term “heterocycle” refers to heteroaromatic and heteroalicyclic groups containing one to four ring heteroatoms each selected from O, S and N. In certain instances, each heterocyclic group has from 4 to 10 atoms in its ring system, and with the proviso that the ring of said group does not contain two adjacent O or S atoms. Non-aromatic heterocyclic groups include groups having 3 atoms in their ring system, but aromatic heterocyclic groups must have at least 5 atoms in their ring system. The heterocyclic groups include benzo-fused ring systems. An example of a 3-membered heterocyclic group is aziridinyl (derived from aziridine). An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydropyranyl, tetrahydrothienyl, tetrahydropyranyl, dihydropyranyl, tetrahydrothiopyranyl, piperidine, morpholino, thiomorpholino, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclohexanyl, 3-azabicycloheptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The terms “heteroaryl” or, alternatively, “heteroaromatic” refers to an aryl group that includes one or more ring heteroatoms selected from nitrogen, oxygen and sulfur. In certain embodiments, heteroaryl groups are monocyclic or polycyclic. Illustrative examples of heteroaryl groups include the following moieties:

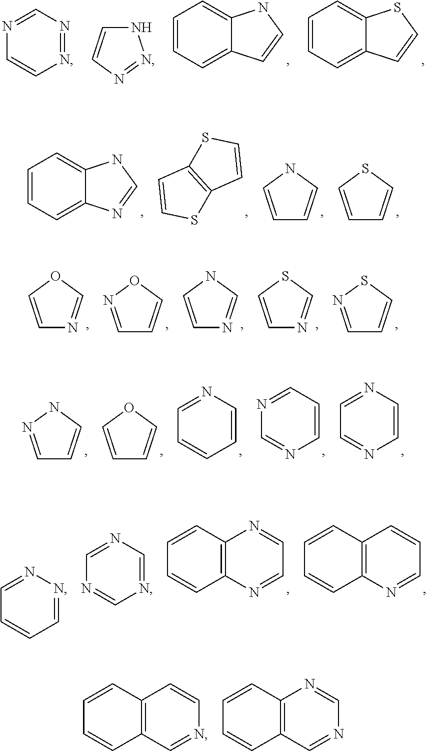

and the like.

A “heteroalicyclic” group or “heterocycloalkyl” group refers to a cycloalkyl group, wherein at least one skeletal ring atom is a heteroatom selected from nitrogen, oxygen and sulfur. In various embodiments, the radicals are with an aryl or heteroaryl. Illustrative examples of heterocycloalkyl groups, also referred to as non-aromatic heterocycles, include:

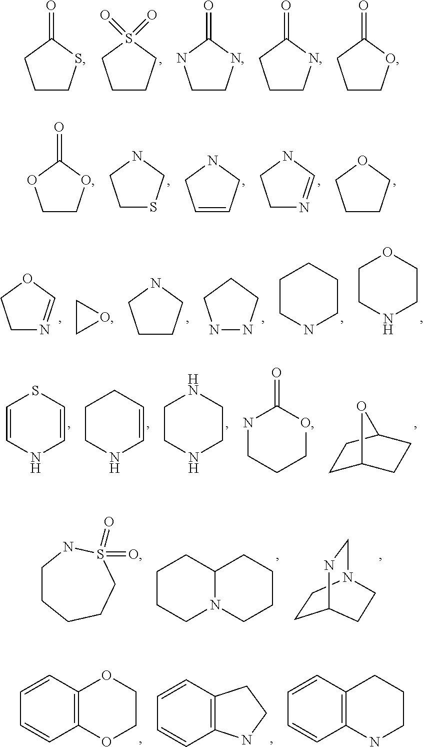

and the like. The term heteralicyclic also includes all ring forms of the carbohydrates, including but not limited to the monosaccharides, the disaccharides and the oligosaccharides.

The term “halo” or, alternatively, “halogen” means fluoro, chloro, bromo and iodo.

The terms “haloalkyl,” and “haloalkoxy” include alkyl and alkoxy structures that are substituted with one or more halogens. In embodiments, where more than one halogen is included in the group, the halogens are the same or they are different.

The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃.

A “cyano” group refers to a —CN group.

An “isocyanato” group refers to a —NCO group.

A “thiocyanato” group refers to a —CNS group.

An “isothiocyanato” group refers to a —NCS group.

“Alkoyloxy” refers to a RC(O)O— group.

“Alkoyl” refers to a RC(O)— group.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Exemplary pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

In one embodiment, the compound of formula I is not the compound of formula II. In certain embodiments, the present invention provides a pharmaceutically acceptable derivative of a compound of the formula II.

In one embodiment, the present invention provides compound of formula I or pharmaceutically acceptable derivative of compound of formula I or II including salts, solvtes, or hydrates for arresting or inhibiting proliferation or obliterating cancer stem cells:

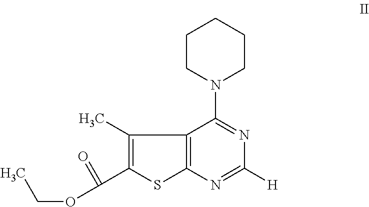 [SMILES: CCOC(=O)c1sc2ncnc(N3CCCCC3)c2c1C]

In one embodiment the present invention provides compound of formula III or formula IV or derivatives thereof.

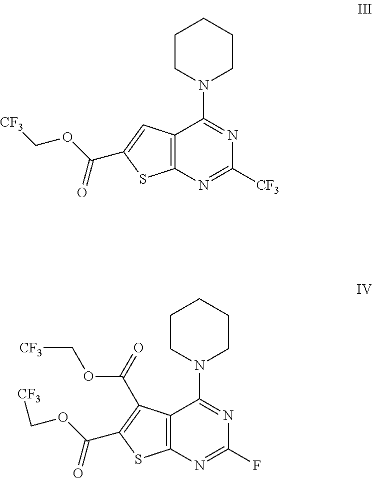 [SMILES: O=C(OCC(F)(F)F)c1cc2c(N3CCCCC3)nc(C(F)(F)F)nc2s1 ; O=C(OCC(F)(F)F)c1sc2nc(F)nc(N3CCCCC3)c2c1C(=O)OCC(F)(F)F]

In some embodiments, provided herein are compositions comprising a therapeutically effective amount of the compound of formula I, or a pharmaceutically acceptable salt or derivative thereof and a pharmaceutically acceptable excipient including carrier, adjuvant, vehicle or mixtures thereof.

In certain embodiments, the present invention provides a composition for arresting or inhibiting proliferation or obliterating cancer stem cells comprising a compound having formula II or a pharmaceutically acceptable salt or derivative thereof and a pharmaceutically acceptable excipient including carrier, adjuvant, or vehicle. The preferable derivative may be a pharmaceutically acceptable ester, or salt of an ester.

The amount of compound in compositions of this invention may be such that it is effective in arresting or inhibiting proliferation or obliterating cancer stem cells, in a biological sample or in a subject in the need thereof. In certain embodiments, the amount of compound in compositions may be such that it is effective to measurably arresting or inhibiting proliferation or obliterating cancer stem cells, in a biological sample or in a subject in the need thereof. In certain embodiments, the composition may comprise between the biologically effective dose and the maximum tolerated dose of the compound of the invention or it's pharmaceutically acceptable salt, ester, or salt of an ester.

In certain embodiments, a composition of this invention may be formulated for administration to a subject in the need thereof. A “subject” is a mammal, preferably a human, but can also be an animal in need of veterinary treatment. The term “subject in the need thereof” refers to a patient suffering from disease, disorder or condition associated with proliferation of cancer stem cells for example any type of cancer or relapse or recurrence of cancer.

The term pharmaceutically acceptable excipient, carrier, adjuvant, or vehicle refers to a non-toxic excipient, carrier, adjuvant, or vehicle that does not destroy the pharmacological activity of the compound with which it is formulated.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or residue thereof.

Compositions of the present invention may be formulated into a suitable dosage form to be administered orally, parenterally, by inhalation spray, topically, rectally, nasally, buccally, vaginally or via an implanted reservoir. Compositions of the present invention may be formulated into dosage forms including liquid, solid, and semisolid dosage forms. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. Preferably, the compositions are administered orally, intravenously or intraperitoneally.

Sterile injectable forms of the compositions of this invention may be sterile injectable aqueous solution or oleaginous suspension in a non-toxic parenterally acceptable diluent or solvent, or suspension, suitable dispersing or wetting agents and suspending agents.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. Depot injectable formulations may also be prepared by entrapping the compound in liposomes or microemulsions that are compatible with body tissues.

Pharmaceutically acceptable compositions of this invention may be orally administered in any orally acceptable dosage form including, but not limited to, capsules, tablets, aqueous suspensions or solutions.

Solid dosage forms for oral administration include but are not limited to capsules, tablets, pills, powders, and granules. In such solid dosage forms, the active compound is mixed with at least one inert, pharmaceutically acceptable excipient or carrier, fillers or extenders, binders, humectants, disintegrating agents, solution retarding agents, absorption accelerators, wetting agents, absorbents, lubricants, buffering agents, and/or mixtures thereof.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and/or emulsifiers. Besides inert diluents, the oral compositions can also include adjuvants such as wetting agents, emulsifying and suspending agents, sweetening, flavoring, and perfuming agents.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For topical applications, provided pharmaceutically acceptable compositions may be formulated in a suitable ointment containing the active component suspended or dissolved in one or more carriers. Alternatively, provided pharmaceutically acceptable compositions can be formulated in a suitable lotion or cream containing the active components suspended or dissolved in one or more pharmaceutically acceptable carriers.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present invention contemplates the use of transdermal patches, which may have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Ophthalmic formulation, ear drops, and eye drops are also contemplated as being within the scope of this invention. For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative. Alternatively, for ophthalmic uses, the pharmaceutically acceptable compositions may be formulated in an ointment.

Compositions of this invention may also be administered by nasal aerosol or inhalation. Such compositions are prepared according to techniques well-known in the art of pharmaceutical formulation and may be prepared as solutions in suitable preservatives, absorption promoters to enhance bioavailability, and/or other conventional solubilizing or dispersing agents.

Topical application for the lower intestinal tract can be effected in a rectal suppository formulation (see above) or in a suitable enema formulation. Topically-transdermal patches may also be used. Compositions for rectal or vaginal administration are preferably suppositories which can be prepared by mixing the compounds of this invention with suitable non-irritating excipients or carriers.

Most preferably, pharmaceutically acceptable compositions of this invention may be formulated for oral administration. Such formulations may be administered with or without food.

The amount of compounds of the present invention that may be combined with the pharmaceutically acceptable excipient or carriers to produce a composition in a single dosage form will vary depending upon the subject to be treated, the particular mode of administration. Preferably, provided compositions should be formulated so that a effective dosage of the compound of the invention can be administered to a subject receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In one embodiment compounds having the general formula I or a pharmaceutically acceptable salt thereof or compositions thereof may be used for arresting or inhibiting proliferation or obliterating cancer stem cells and thereby treating associated disorders or diseases or conditions. Thus, provided compounds may be useful for treating cancers, including, but not limited to hematological cancers and solid tumors.

In certain embodiments compounds of the present invention having the formula II or a pharmaceutically acceptable salt thereof or compositions thereof may be used for arresting or inhibiting proliferation or obliterating cancer stem cells and thereby treating associated disorders or diseases or conditions. Thus, provided compounds are useful for treating cancers, including, but not limited to hematological cancers and solid tumors.

As used herein, the term “cancer stem cell” includes any cell characterized by the ability to undergo mitotic division and differentiate into one or more types of cell found in a neoplasm. “Cancer stem cells” include any cell that is totipotent, pluripotent, multipotent, oligopotent, or unipotent, “Cancer stem cells” include progenitor cells.

As used herein, the terms “arresting or inhibiting proliferation or obliterating cancer stem cells” refer to the arresting or inhibiting proliferation or obliteration of cancer stem cells by inhibiting or suppressing growth, division, maturation or viability of cancer stem cells, and/or causing the death of cancer stem cells, individually or in aggregate with other cancer stem cells, by cytotoxicity or the induction of apoptosis.

In another embodiment the present invention provides a method of arresting or inhibiting proliferation or obliterating cancer stem cells by administering compounds having the general formula I or a pharmaceutically acceptable salt or derivative thereof or compositions comprising the same in subjects in the needthereof.

In certain embodiments the present invention provides a method of arresting or inhibiting proliferation or obliterating cancer stem cells by administering the compound having formula II or a pharmaceutically acceptable salt thereof or compositions comprising the same in subjects in the needthereof.

Without being bound by a particular theory or mechanism, the arresting or inhibiting proliferation or obliterating cancer stem cells population arrests or inhibits proliferation or obliterates the cancer cell population produced by the cancer stem cell population, and thus, arrest, inhibits or obliterates the growth of a tumor, the bulk size of a tumor, the formation of a tumor and/or the formation of metastases. In other words, the arresting or inhibiting proliferation or obliterating cancer stem cells population prevents the formation, reformation or growth of a tumor and/or metastases by cancer cells.

In certain embodiments, the methods of the present invention may be designed to result in a concentration (e.g., in blood, plasma, serum, tissue, and/or tumor) of a therapy(ies) that will stabilize or reduce a cancer stem cell population.

Since cancer stem cells often make up only a subpopulation of a tumor, a therapy that stabilizes, reduces or eliminates cancer stem cells may require a longer period of time than is traditionally expected for a cancer patient to achieve arresting or inhibiting proliferation or obliterating cancer stem cells growth, size and/or formation of a tumor and/or metastases, or an amelioration of cancer-related symptoms. Accordingly, during this additional time period, there is an opportunity to deliver additional therapy, albeit at less toxic (e.g., lower) doses. As a result of arresting or inhibiting proliferation or obliterating cancer stem cells population, the cancer may be significantly impaired, the frequency of responses increased albeit potentially occurring at later time points, the duration of a remission increased, and/or the frequency particular embodiment, the reduction in the cancer stem cell population may be determined by a method as described herein.

The activity of a compound utilized in this invention for eradicating or inhibiting proliferation of cancer stem cells or other cancer cells, may be assayed in vitro or in vivo. An in vivo assessment of the eliminating or cytotoxic activity of the compounds of the invention may be made using an animal model of cancer, e.g., a rodent or primate model. Cell-based assays may be performed using, e.g., a cell line isolated from a tumor or blood-borne cancer. Cell-based assays for activity against a specific protein or nucleic acid component of a cancer cell line. e.g., an enzyme, structural protein, cell surface markers, DNA or RNA, or microarrays, may also be performed. Additionally, biochemical or mechanism-based assays, e.g., transcription assays using a purified protein, Northern blot, RT-PCR, etc., may be performed. In vitro assays include assays that determine cell morphology, viability, cell count, or growth inhibition, and/or the cytotoxicity, enzyme inhibitory activity, and/or the subsequent functional consequences of treatment of cancer cells with compounds of the invention. Alternate in vitro assays quantitate the ability of the compounds of the present invention to bind to protein or nucleic acid molecules within the cell.

Examples of cancer cell lines which may be used for testing or whose proliferation may be arrested or inhibited or obliterated by the compounds and compositions described herein and against which the methods described herein may be useful include but are not limited to LNCaP, MDA MB 231, MCF7, DU145, PC3, T47D, HeLa, or other cell lines derived from tissues including, but not limited to, prostate, breast, fibroblast, cervical, kidney, colon, pancreas or lung.

According to one embodiment, the invention relates to a method of arresting or inhibiting proliferation or obliterating cancer stem cells in a biological sample comprising the step of contacting said biological sample with a compound of formula I or II or derivative thereof or composition comprising the same in an effective amount. In certain embodiments, the invention relates to a method of killing cancer cells or cancer stem cells in a biological sample comprising the step of contacting said biological sample with a compound of formula I or II or derivative thereof or composition comprising the same in an effective amount.

The term “compound of this invention” or “compound of the invention”, as used herein, includes the compounds having the general formula I, or a pharmaceutically acceptable salt or derivative of compound of formula I or II.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Eradicating cancer stem cells in a biological sample may be useful for a variety of purposes that are known to one of skill in the art. Examples of such purposes include, but are not limited to biological assays, gene expression studies, and biological target identification.

In one more embodiment the present invention provides a method of treatment of disorders or diseases or conditions associated with cancer stem cells by administering compounds of formula I or II or derivative thereof or compositions comprising the same in subjects in the need thereof.

In certain embodiments, the present invention provides a method for treating a disorder mediated by cancer stem cells in a patient in need thereof, comprising the step of administering to said patient a compounds of formula I or II or derivative thereof or composition comprising the same in an effective amount. Such disorders include cancer or recurrence or relapse of cancer or other proliferative diseases.

In certain embodiments, the invention relates to a method of eradicating arresting or inhibiting proliferation or obliterating cancer stem cells in a patient, leading to remission of the cancer, comprising the step of administering to said patient a compound of formula I or II or derivative thereof or composition comprising the same in an effective amount.

In some embodiments the compounds of formula I or II or derivative thereof or composition comprising the same in an effective amount may be used in a method of treating a cancer or other proliferative disorder. In some embodiments the present invention provides a method of treating a cancer or other proliferative disorder, comprising administering a compound or composition of the present invention to a patient with a cancer or other proliferative disorder.

In certain embodiments the compounds of formula I or II or derivative thereof or composition comprising the same in an effective amount may be used to treat a cancer in a mammal. In certain embodiments the mammal is a human patient.

In certain embodiments the compounds of formula I or II or derivative thereof or composition comprising the same in an effective amount may be used to treat a cancer in a human patient, said cancer occurring in the patient's prostate, breast, neck, colon, skin, liver, stomach, pancreas, kidney, ovary, lung, testicle, penis, thyroid, parathyroid, pituitary, thymus, retina, uvea, conjunctiva, spleen, head, trachea, gall bladder, rectum, salivary gland, adrenal gland, throat, esophagus, lymph nodes, sweat glands, sebaceous glands, muscle, heart, brain, blood, or bone marrow.

Depending upon the particular condition, or disease, to be treated, additional therapeutic agents, which are normally administered to treat that condition, may be administered in combination with compounds and compositions of this invention. In some embodiments, a provided compound of this invention, or composition thereof, is administered in combination with one or more other chemotherapeutic agents. Such chemotherapeutic agents include, but are not limited to agents such as kinase inhibitors, alkylating agents, anti-metabolites, tubulin stabilizers, tubulin assembly inhibitors, DNA replication inhibitors, cell cycle inhibitors, topoisomerase inhibitors, cytotoxic antibiotics or nanoparticle or protein conjugates of any of the aforementioned agents.

In certain embodiments, a combination of 2 or more chemotherapeutic agents may be administered together with compounds of the invention. In certain embodiments, a combination of 3 or more chemotherapeutic agents may be administered with compounds of the invention. In some embodiments, the chemotherapeutic agents are selected from alkylating agents or anti-metabolites.

Other examples of agents compounds of this invention may also be combined with include, without limitation: vitamins and nutritional supplements, cancer vaccines, antisense agents, a monoclonal or polyclonal antibody, an siRNA therapeutic or other agents for treatments of conditions, disorders or diseases other than cancer.

In one embodiment, such other agent includes one or more anti-proliferative agents, anti-inflammatory agents, immunomodulatory agents or immunosuppressive agents.

Those additional agents may be administered separately from the compound of the invention-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another. The amount of both, the compound of this invention and additional therapeutic agent (in those compositions which comprise an additional therapeutic agent as described above) that may be combined with the carrier materials to produce a single dosage form will vary depending upon the host treated and the particular mode of administration.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent.

The amount of additional therapeutic agent present in the compositions of this invention may be no more than the amount that would normally be administered in a composition comprising that therapeutic agent as the only active agent. Preferably the amount of additional therapeutic agent in the presently disclosed compositions may range from about 5% to 90% of the amount normally present in a composition comprising that agent as the only therapeutically active agent.

Resistance to chemotherapeutic drugs is a major factor limiting the efficacy of therapies against many cancers and other proliferative disorders. The rapid division rate of these cells allows for the development of mutations or upregulation of pumps such as MDR that afford resistance to current first line chemotherapy drugs. The problem of relapse of cancers in a more drug-resistant form is a critical hurdle faced in drug development of new chemotherapeutic drugs to treat cancer patients.

The present invention can address this problem by providing the compounds of this invention and compositions thereof for arresting or inhibiting proliferation or obliterating cancer stem cells and thereby treating associated disorders or diseases or conditions in particular for avoiding or minimizing problem of relapse of cancers.

The compounds of the invention may be prepared according to the methods of synthesis that may be known to one of ordinary skilled in the art or can be specifically designed to synthesize compounds of the invention or their subclasses or species of each of these compounds, as described herein.

The foregoing description of the invention has been set merely to illustrate the invention and is not intended to be limiting. Since modifications of the disclosed embodiments including those relating to the chemical structures, substituents, derivatives, intermediates, syntheses, compositions, formulations and/or methods of use provided herein without departing from the spirit and substance of the invention may occur to person skilled in the art, the invention should be construed to include everything within the scope of the disclosure.

### EXAMPLES

### Example 1

### Preparation of Ethyl-5,6-dimethyl-4-(piperidin-1-yl)thieno[2,3-d]pyrimidine

Three-neck round bottom flask was arranged with water condenser, thermometer pocket on magnetic stirrer and charged ethylacetoacetate (4 ml), malononitrile (2.48 gm), sulfur (1.2 gm) in methanol (37.5 ml) and morpholine (6.97 ml) under stirring at room temperature. The mixture was stirred at room temperature for 10 min. and then refluxed for 3 hours. The reaction was monitored on TLC, after complete conversion reaction mass was allowed to cool at room temperature and filtered under vacuum and the compound obtained as washed with methanol to obtained ethyl 5-amino-4-cyano-3-methylthiophene-2-carboxylate.

Reaction was set as described above and charged ethyl 5-amino-4-cyano-3-methylthiophene-2-carboxylate (0.210 gm, 1 mmol), to which was added 10 ml mixture of formic acid:Conc. Hydrochloric acid (1:1) and refluxed on water bath for 2 hrs. The reaction was monitored on TLC after completion of reaction, allowed to cool at room temperature and poured into crushed ice. Solid obtained was filtered under vacuum and washed with water to get pure product of ethyl 5-methyl-4-oxo-3,4-dihydrothieno[2,3-d]pyrimidine-6-carboxylate.

Similar to above the reaction was set and charged ethyl 5-methyl-4-oxo-3,4-dihydrothieno[2,3-d]pyrimidine-6-carboxylate (0.238 gm 1 mmol), POCL₃ 10 ml and 2 drops of DMF under stirring. When addition was complete, reaction was refluxed for 1 hr, cooled and poured onto crushed ice. The product ethyl 4-chloro-5-methyl thieno[2,3-d]pyrimidine-6-carboxylate was got precipitated out, which was filtered under vacuum and washed with water and dried to get pure ethyl 4-chloro-5-methyl thieno [2,3-d]pyrimidine-6-carboxylate.

Thus obtained pure ethyl 4-chloro-5-methyl thieno [2,3-d]pyrimidine-6-carboxylate (0.256 gm, 1 mmol) was taken in 50 ml round-bottom flask containing 10 ml ethanol. Flask was arranged on magnetic stirrer which was equipped with water condenser, thermometer pocket and charged slowly with Piperidine (0.1 ml, 1 mmol) and refluxed for 4 hrs. After completion of reaction, the reaction mixture was allowed to cool at room temperature and poured onto crushed ice, product ethyl 5-methyl-4-(piperidin-1-yl)thieno[2,3-d]pyrimidine-6-carboxylate got precipitated out which was separated by filtration under vacuum washed with water and dried to get pure product.

### Example 2: In Vitro Colorimetric Cell Death Assay

For assessing cell viability in vitro colorimetric cell death assay was carried out where the cells were grown in two-dimensional surface as follows.

Cancer cells with plating efficiency as mentioned herein below for respective cell lines were plated in a 96 well plate. The plate was incubated for 24 hours in a 5% CO₂ atmosphere at 37 degrees Celsius, a range of concentrations from 10⁻³ M to 10⁻⁶ M of the compound Ethyl-5,6-dimethyl-4-(piperidin-1-yl)thieno[2,3-d]pyrimidine was added to the wells, the plates were incubated further for 48 hours in a 5% CO₂ atmosphere, the plate was centrifuged twice at 3000 rpm for 3 minutes, the supernatant fluid was discarded, 100 uL of 0.5 mg/mL 3-(4,5-Dimethylthiazol-2-yl)-2,5-Diphenyltetrazolium Bromide (MTT) solution was added and the plate was incubated for 4 hours in a 5% CO₂ atmosphere at 37 degrees Celsius. The plate was then centrifuged twice at 3000 rpm for 3 minutes, supernatant was aspirated very carefully, 200 uL Dimethyl sulfoxide (DMSO) was added to each well to solubilize MTT crystals and mixed well by shaking the plate, the plate was incubated for 10 minutes in a 5% CO₂ atmosphere at 37 degrees Celsius, the plate was placed on the shaker of an ELISA plate reader and the absorbance at 570 nm was measured, the percentage of viable cells remaining was calculated by first subtracting the background absorbance then comparing to the absorbance of a non-drug-treated cell sample, and the results were plotted on a graph to determine the IC50 for the compound Ethyl-5,6-dimethyl-4-(piperidin-1-yl)thieno[2,3-d]pyrimidine which was calculated by regression analysis.

The results of the in vitro colorimetric cell death assays on different cancer cell lines are given below in Table1, Table2, Table3, Table4, Table5 and Table6.

TABLE 1
Colorimetric cell death assay data of compound Ethyl-5,6-dimethyl-
4-(piperidin-1-yl)thieno[2,3-d]pyrimidine and Cisplatin
(standard therapeutic drug) on Prostate cancer cell lines.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Prostate cancer | Efficiency | (IC 50 Value | (IC 50 Value in |
| cell lines | per 200 uM/well | in uM) | uM) |
| PC3 | 10,000 | 11.22 | 1.479 |
| DU145 | 5000 | 31.62 | 2.82 |
| LNCaP | 10,000 | 52.5 | 9.55 |

The above result shows that the compound Ethyl-5,6-dimethyl-4-(piperidin-1-yl)thieno[2,3-d]pyrimidine exhibit higher anticancer activity in prostate cancer cell lines than Cisplatin (standard therapeutic drug) indicating better activity of compound Ethyl-5,6-dimethyl-4-(piperidin-1-yl)thieno[2,3-d]pyrimidine on prostate cancer cells.

TABLE 2
Colorimetric cell death assay data of compound Ethyl-5,6-dimethyl-
4-(piperidin-1-yl)thieno[2,3-d]pyrimidine and Cisplatin
(standard therapeutic drug) on Breast cancer cell lines.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Breast cancer | Efficiency per | (IC 50 Value | (IC 50 Value in |
| cell lines | 200 uM/well | in uM) | uM) |
| MDAMB231 | 10,000 | 48.42 | 1.96 |
| MCF-7 | 7500 | 29.24 | 1.20 |
| T47D | 20,000 | 58.9 | 6.31 |

The above shows that the compound Ethyl-5,6-dimethyl-4-(piperidin-1-yl)thieno[2,3-d]pyrimidine exhibits higher anticancer activity in Breast Cancer Cell line than Cisplatin (standard therapeutic drug) indicating that Ethyl-5,6-dimethyl-4-(piperidin-1-yl)thieno[2,3-d]pyrimidine has better activity on breast cancer cells.

TABLE 3
Colorimetric cell death assay data of compound Ethyl-5,6-dimethyl-
4-(piperidin-1-yl)thieno[2,3-d]pyrimidine and Cisplatin
(standard therapeutic drug) on Fibroblast cancer cell line.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Fibroblast cancer | Efficiency | (IC 50 Value | (IC 50 Value in |
| cell line | per 200 uM/well | in uM) | uM) |
| L929 | 5000 | 29.005 | 3.53 |

The above shows that the compound Ethyl-5,6-dimethyl-4-(piperidin-1-yl)thieno[2,3-d]pyrimidine exhibits higher anticancer activity in Fibroblast Cancer Cell line than Cisplatin (standard therapeutic drug) indicating that the compound Ethyl-5,6-dimethyl-4-(piperidin-1-yl)thieno[2,3-d]pyrimidine has better activity on Fibroblast cancer cells.

TABLE 4
Colorimetric cell death assay data of compound Ethyl-5,6-dimethyl-
4-(piperidin-1-yl)thieno[2,3-d]pyrimidine and Cisplatin
(standard therapeutic drug) on Cervical cancer cell line.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Cervical cancer | Efficiency | (IC 50 Value | (IC 50 Value in |
| cell line | per 200 uM/well | in uM) | uM) |
| HeLa | 5000 | 33.9 | 8.27 |
| SiHa | 10000 | 35.48 | 6.08 |
| Bu25tk | 7000 | 59.27 | 11.73 |

The above shows that the compound Ethyl-5,6-dimethyl-4-(piperidin-1-yl)thieno[2,3-d]pyrimidine exhibits higher anticancer activity in Cervical Cancer Cell line than Cisplatin (standard therapeutic drug) indicating that the compound Ethyl-5,6-dimethyl-4-(piperidin-1-yl)thieno[2,3-d]pyrimidine has better activity on Cervical cancer cells.

TABLE 5
Colorimetric cell death assay data of compound Ethyl-5,6-dimethyl-
4-(piperidin-1-yl)thieno[2,3-d]pyrimidine and Cisplatin
(standard therapeutic drug) on Colon cancer cell line.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Colon cancer | Efficiency | (IC 50 Value | (IC 50 Value in |
| cell line | per 200 uM/well | in uM) | uM) |
| Colo320 | 15000 | 38.34 | 7.91 |

The above shows that the compound Ethyl-5,6-dimethyl-4-(piperidin-1-yl)thieno[2,3-d]pyrimidine exhibits higher anticancer activity in Colon Cancer Cell line than Cisplatin (standard therapeutic drug) indicating that the compound Ethyl-5,6-dimethyl-4-(piperidin-1-yl)thieno[2,3-d]pyrimidine has better activity on Colon cancer cells.

TABLE 6
Colorimetric cell death assay data of compound Ethyl-5,6-dimethyl-
4-(piperidin-1-yl)thieno[2,3-d]pyrimidine and Cisplatin
(standard therapeutic drug) on Hepatic cancer cell line.

| | | | Ethyl-5,6- |
|---|---|---|---|
| | | | dimethyl-4- |
| | | | (piperidin-1- |
| | | | yl)thieno[2,3- |
| | Plating | Cisplatin | d]pyrimidine |
| Hepatic cancer | Efficiency | (IC 50 Value | (IC 50 Value in |
| cell line | per 200 uM/well | in uM) | uM) |
| Hep3B | 10000 | 37.58 | 13.26 |

The above shows that the compound Ethyl-5,6-dimethyl-4-(piperidin-1-yl)thieno[2,3-d]pyrimidine shows high anticancer activity in Hepatic Cancer Cell line than Cisplatin (standard therapeutic drug) indicating that the compound Ethyl-5,6-dimethyl-4-(piperidin-1-yl)thieno[2,3-d]pyrimidine has better activity on Hepatic cancer cells.

### Example 3: 3D Primary Sphere Assay

The ability of cancer stem cells to form spheres in a serum free media was determined by 3D Primary Sphere Assay, where the spheres were in a suspended form. Through this assay the potency of the compound Ethyl-5,6-dimethyl-4-(piperidin-1-yl)thieno[2,3-d]pyrimidine to kill cancer stem cells in comparison to the standard chemotherapeutic drug such as Cisplatin was evaluated as follows.

The Cells were grown in three dimensions on a plastic substrate, harvested in suspension in serum-free media, then the cells in the sample were trypsinised and a single cell suspension was formed by passing through a cell strainer. The cells were diluted according to the predetermined plating efficiency for the cell line being studied by suspending the cells in stem cell culture medium. 100 uL of this suspension was added into each well of a 96 well suspension plate, and the plate was incubated at 37 degrees Celsius in 5% CO₂ atmosphere for 24 hours, then 2 uL of appropriate concentrations of the drugs were added into each respective well along with 100 uL of stem cell culture medium, and the plates incubated at 37 degrees Celsius under 5% CO₂ atmosphere for 72 hours. 2.5 uL of the appropriate drug concentration was added to each respective well along with 50 uL of stem cell culture medium and the plates were incubated at 37 degrees Celsius under 5% CO₂ atmosphere for 72 hours. 3 uL of the appropriate concentration of the compound as mentioned below was added to each respective well along with 50 uL of stem cell culture medium, incubated at 37 degrees Celsius under 5% CO₂ atmosphere for 72 hours, the spheres formed were observed under a microscope, counted and scored by size.

Results of the in vitro 3D sphere forming stem cell assay were set forth in a tabular manner. The number in each box showed the total number of spheres formed in the presence of either Cisplatin or compound of Invention Ethyl-5,6-dimethyl-4-(piperidin-1-yl)thieno[2,3-d]pyrimidine at each drug concentration. GC refers to a growth control performed in the absence of drug or solvent (DMSO). GCD refers to a growth control performed in the absence of drug, but in the presence of DMSO.

TABLE 7
3D Primary Sphere Assay data of the compound Ethyl-5,6-dimethyl-4-(piperidin-
1-yl)thieno[2,3-d]pyrimidine (plating efficiency 2500 cells/100 μL/well)
and Cisplatin (standard therapeutic drug) for Prostate cancer cell line PC3.

| | Dilution | | | | | | |
|---|---|---|---|---|---|---|---|
| | (from stock | | | | | | |
| Cell | of 0.5M) | 10 | 100 | 1000 | 10,000 | | |
| Line | Final conc. | 1250 μM | 125 μM | 12.5 μM | 1.25 μM | GC | GCD |
| PC3 | Cisplatin | 74(±7) | 88(±6) | 107(±9) | 109(±6) | 120(±10) | 103(±9) |
| | Ethyl-5,6- | 0(±0) | 14(±3) | 28(±4) | 48(±4) | 120(±10) | 103(±9) |
| | dimethyl-4- | | | | | | |
| | (piperidin- | | | | | | |
| | 1-yl) thieno | | | | | | |
| | [2,3-d] | | | | | | |
| | pyrimidine | | | | | | |

GC: Growth Control,
GCD: Growth Control with DMSO

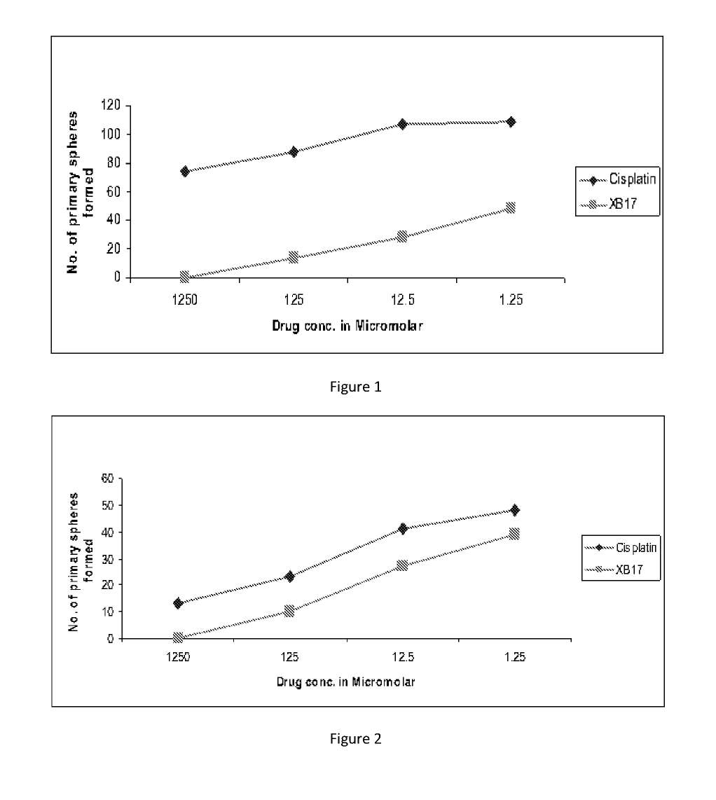

The above Table 7 and FIG. 1 shows that the compound Ethyl-5,6-dimethyl-4-(piperidin-1-yl)thieno[2,3-d]pyrimidine has better anticancer effect on primary spheres of PC3 as compared to cisplatin.

PC3 is a highly metastatic Prostate cancer cell line. The compound Ethyl-5,6-dimethyl-4-(piperidin-1-yl)thieno[2,3-d]pyrimidine showed better anticancer activity in primary spheres of Prostate Cancer Cell line (PC3) than Cisplatin (standard therapeutic drug), indicating that the compound Ethyl-5,6-dimethyl-4-(piperidin-1-yl)thieno[2,3-d]pyrimidine is more potent on the primary spheres of Prostate cancer cells than Cisplatin as the number of primary spheres formed by these cancer cells are significantly less in Ethyl-5,6-dimethyl-4-(piperidin-1-yl)thieno[2,3-d]pyrimidine than those formed in Cisplatin as can be seen in FIG. 1.

TABLE 8
3D Primary Sphere Assay data of the compound Ethyl-5,6-dimethyl-4-(piperidin-
1-yl)thieno[2,3-d]pyrimidine (plating efficiency 1000 cells/100 μL/well)
and Cisplatin (standard therapeutic drug) for Prostate cancer cell line DU145.

| | Dilution | | | | | | |
|---|---|---|---|---|---|---|---|
| | (from stock | | | | | | |
| Cell | of 0.5M) | 10 | 100 | 1000 | 10,000 | | |
| Line | Final conc. | 1250 μM | 125 μM | 12.5 μM | 1.25 μM | GC | GCD |
| DU145 | Cisplatin | 12(±3) | 23(±4) | 41(±5) | 48(±5) | 49(±5) | 41(±4) |
| | Ethyl-5,6- | 0(±0) | 10(±2) | 27(±6) | 39(±5) | 49(±5) | 41(±4) |
| | dimethyl-4- | | | | | | |
| | (piperidin- | | | | | | |
| | 1-yl) thieno | | | | | | |
| | [2,3-d] | | | | | | |
| | pyrimidine | | | | | | |

GC: Growth Control,
GCD: Growth Control with DMSO

The above Table 8 and FIG. 2 shows that the compound Ethyl-5,6-dimethyl-4-(piperidin-1-yl)thieno[2,3-d]pyrimidine has better anticancer effect on primary spheres of DU145 compared to Cisplatin.

DU145 is a moderate metastatic Prostate cancer cell line. The compound Ethyl-5,6-dimethyl-4-(piperidin-1-yl)thieno[2,3-d]pyrimidine shows high anticancer activity in primary spheres of Prostate Cancer Cell line (DU145) than Cisplatin (standard therapeutic drug), indicating that the compound Ethyl-5,6-dimethyl-4-(piperidin-1-yl)thieno[2,3-d]pyrimidine is more potent on the primary spheres of Prostate cancer cells than Cisplatin as the number of primary spheres formed by these cancer cells are significantly less in Ethyl-5,6-dimethyl-4-(piperidin-1-yl)thieno[2,3-d]pyrimidine than those formed in Cisplatin as can be seen in FIG. 2.

TABLE 9
3D Primary Sphere Assay data of the compound Ethyl-5,6-dimethyl-4-(piperidin-
1-yl)thieno[2,3-d]pyrimidine (plating efficiency 2000 cells/100 μL/well)
and Cisplatin (standard therapeutic drug) for Breast cancer cell line MDA MB.

| | Dilution | | | | | | |
|---|---|---|---|---|---|---|---|
| | (from stock | | | | | | |
| Cell | of 0.5M) | 10 | 100 | 1000 | 10,000 | | |
| Line | Final conc. | 1250 μM | 125 μM | 12.5 μM | 1.25 μM | GC | GCD |
| MDA | Cisplatin | 25(±5) | 39(±3) | 52(±6) | 76(±8) | 92(±7) | 83(±6) |
| MB231 | Ethyl-5,6- | 0(±0) | 0(±0) | 9(±2) | 28(±4) | 92(±7) | 83(±6) |
| | dimethyl-4- | | | | | | |
| | (piperidin- | | | | | | |
| | 1-yl) thieno | | | | | | |
| | [2,3-d] | | | | | | |
| | pyrimidine | | | | | | |

GC: Growth Control,
GCD: Growth Control with DMSO

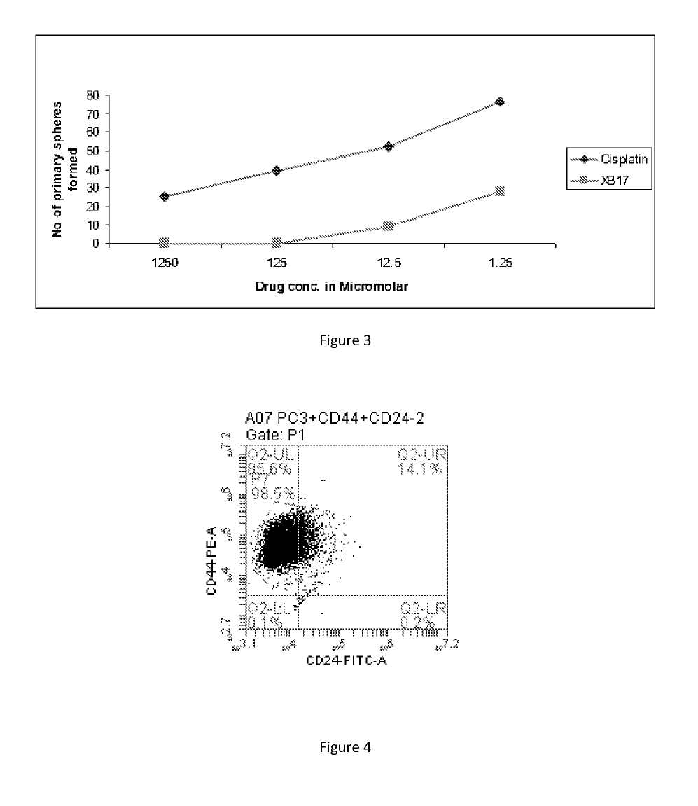

The above Table 9 and FIG. 3 shows that the compound Ethyl-5,6-dimethyl-4-(piperidin-1-yl)thieno[2,3-d]pyrimidine has better anticancer effect on primary spheres of MDA MB compared to Cisplatin.

MDAMB231 is a highly metastatic breast cancer cell line with high stem cell population compared to other cell lines. The compound of Ethyl-5,6-dimethyl-4-(piperidin-1-yl)thieno[2,3-d]pyrimidine shows high anticancer activity in primary spheres of Breast Cancer Cell line (MDAMB 231) than cisplatin (standard therapeutic drug), indicating that the compound Ethyl-5,6-dimethyl-4-(piperidin-1-yl)thieno[2,3-d]pyrimidine is more potent on the primary spheres of Breast cancer cells than cisplatin as the number of primary spheres formed by these cancer cells are significantly less in Ethyl-5,6-dimethyl-4-(piperidin-1-yl)thieno[2,3-d]pyrimidine than those formed in Cisplatin as can be seen in FIG. 3.

### Example 4: Flow Cytometry Study of Effect of Ethyl-5,6-dimethyl-4-(piperidin-1-yl)thieno[2,3-d]pyrimidine and Cisplatin on Prostate Cancer Cell Line PC3

For Flow cytometry assay the PC3 cancer cells (0.35×106) were cultured in Dulbecco's Modified Eagle's medium (DMEM) with 10% Fetal Bovine Serum (F.B.S) in 60 mm tissue culture plates for 24 hrs in 5% CO2 at 37° C. Cells were then exposed to IC25 drug conc. of Cisplatin in duplicates. Similarly other sets of cells were treated with IC25 drug conc. of Ethyl-5,6-dimethyl-4-(piperidin-1-yl)thieno[2,3-d]pyrimidine for 48 hrs. and incubated in 5% CO2 at 37° C. for 48 hrs. Appropriate controls that is growth control (cells+DMEM medium) and solvent control (cells+control DMEM medium+DMSO) and medium control (DMEM) were also kept along with the experimental sets. After 48 hrs the cells were observed under the microscope, trypsinised, washed with Dulbecco's phosphate-buffered saline (D.P.B.S.) 50 μl of cells were taken for each set and 5 μl of each of the CD44-PE labeled and CD24-FITC labeled antibodies were added. The sets were incubated for 45 mins at 4° C. for proper binding of antibodies. After incubation the cells were washed with 200 μl of D.P.B.S by centrifugation. The supernatant was discarded and the cells were finally suspended in 300 μl of FACS buffer (4% Fetal Bovine Serum in D.P.B.S). Samples were kept at 4° C. in dark till they were acquired on FACS. Acquisition was done on BD-FACS Accuri C6.

1. Untreated Population: PC3 cells without drug treatment were stained with anti-CD44-PE labeled and anti-CD24-FITC labeled antibodies and the expression was observed in the quadrant plot 85.61% cells (UL) (FIG. 4) expressed CD44 indicating a cell population rich with cancer stem cells.

2. FACS Results for PC3 cells exposed to IC25 drug conc. of Cisplatin for 48 hrs: Exposure of Cisplatin IC25 drug conc. did not have much effect on CD44 population of PC3 cells indicating that it is not very effective on cancer stem cells (FIG. 5).

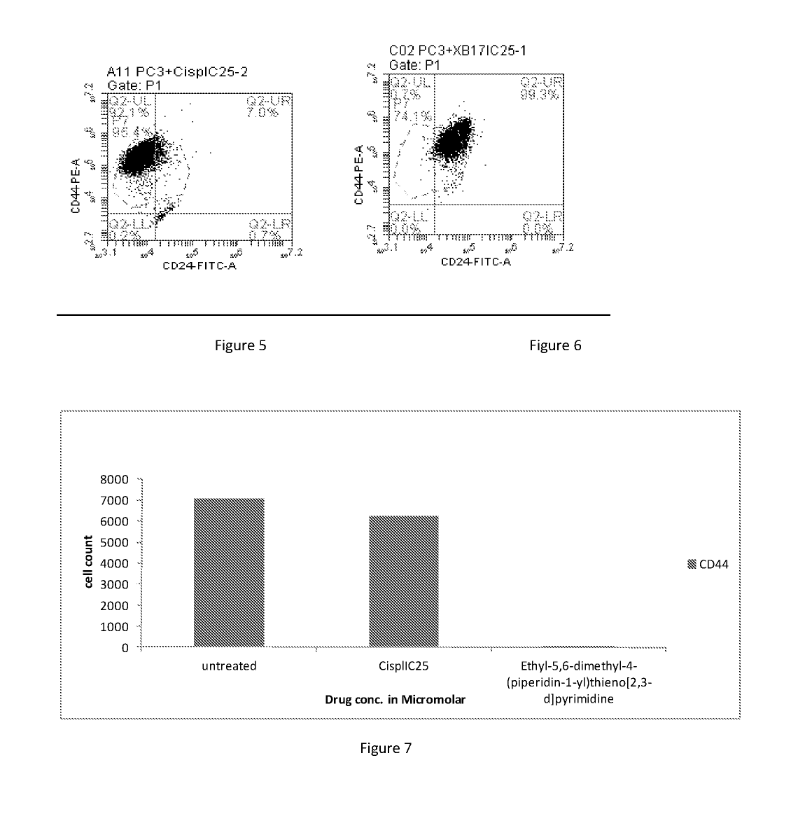

3. FACS Results for PC3 cells exposed to IC25 drug conc. of Ethyl-5,6-dimethyl-4-(piperidin-1-yl)thieno[2,3-d]pyrimidine for 48 hrs: PC3 cells exposed to IC25 drug conc. of Ethyl-5,6-dimethyl-4-(piperidin-1-yl)thieno[2,3-d]pyrimidine showed a drastic effect on CD44 population. There was almost a complete shift from CD44 region to the co-expressed region (FIG. 6) indicating the action of Ethyl-5,6-dimethyl-4-(piperidin-1-yl)thieno[2,3-d]pyrimidine on cancer stem cells of PC3 cells which is a highly metastatic cell line.

The above results as well as FIG. 7 shows that Ethyl-5,6-dimethyl-4-(piperidin-1-yl)thieno[2,3-d]pyrimidine exhibits better activity on cancer stem cells of PC3 compared to standard therapeutic drug Cisplatin.

### Example 5: Flow Cytometry Study of Effect of Ethyl-5,6-dimethyl-4-(piperidin-1-yl)thieno[2,3-d]pyrimidine and Cisplatin on Prostate Cancer Cell Line DU145

Flow cytometry assay was carried out as described above in Example 4, except that in place of the PC3 cancer cells DU145 cancer cells were used.

1. Untreated Population: DU145 cells without drug treatment were stained with anti-CD44-PE labeled and anti-CD24-FITC labeled antibodies and the expression was observed in the quadrant plot. 70.18% cells (UL) (FIG. 8) expressed CD44 indicating a cell population rich with cancer stem cells.

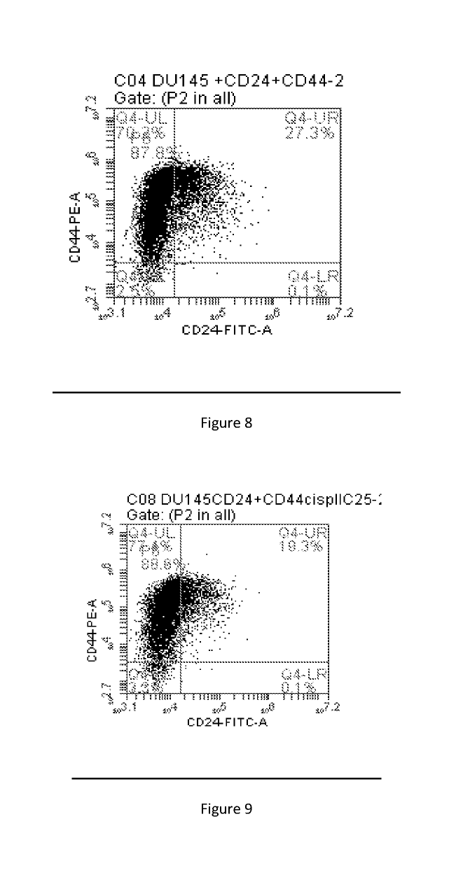

2. FACS Results for DU145 cells exposed to IC25 drug conc. of Cisplatin for 48 hrs: Exposure of Cisplatin IC25 drug conc. did not have much effect on CD44 population of DU145 cells indicating that it is not very effective on cancer stem cells (FIG. 9).

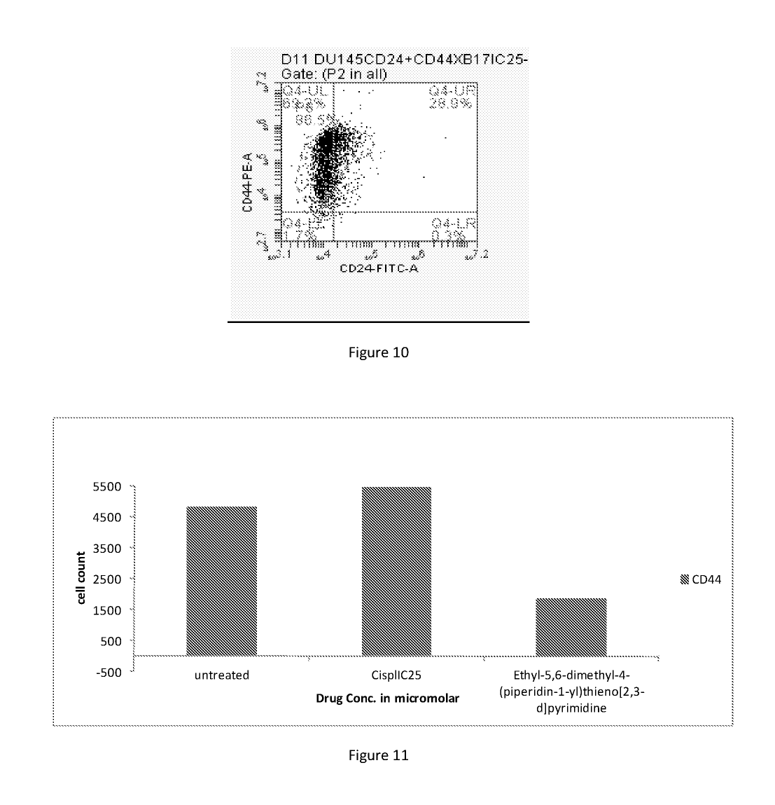

3. FACS Results for DU145 cells exposed to IC25 drug conc. of Ethyl-5,6-dimethyl-4-(piperidin-1-yl)thieno[2,3-d]pyrimidine for 48 hrs: DU145 cells exposed to IC25 drug conc. of Ethyl-5,6-dimethyl-4-(piperidin-1-yl)thieno[2,3-d]pyrimidine showed a drastic effect on CD44 population (FIG. 10), indicating the action of Ethyl-5,6-dimethyl-4-(piperidin-1-yl)thieno[2,3-d]pyrimidine on cancer stem cells of DU145 which is a moderately metastatic cell line.

The above results as well as FIG. 11 shows that Ethyl-5,6-dimethyl-4-(piperidin-1-yl)thieno[2,3-d]pyrimidine exhibits better activity on cancer stem cells of DU145 compared to standard therapeutic drug Cisplatin.

While the invention has been described in conjunction with enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

1. A compound of formula (I) 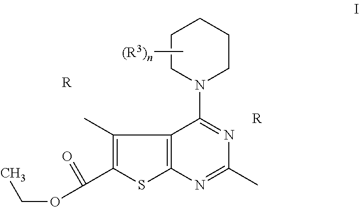
or pharmaceutically acceptable derivatives thereof for arresting or inhibiting proliferation or obliterating cancer stem cells, wherein;
each R¹, R² and R³ is independently selected from halogen, C1-6 haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —OC(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulphur; and
each n is independently 0-5.

2. The compound as claimed in claim 1, wherein the compound is a pharmaceutically acceptable derivative of compound of formula II:

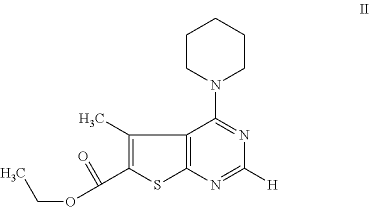 [SMILES: CCOC(=O)c1sc2ncnc(N3CCCCC3)c2c1C]

3. The compound as claimed in claim 2, wherein the pharmaceutically acceptable derivative of compound of formula II is a compound of formula III or formula IV:

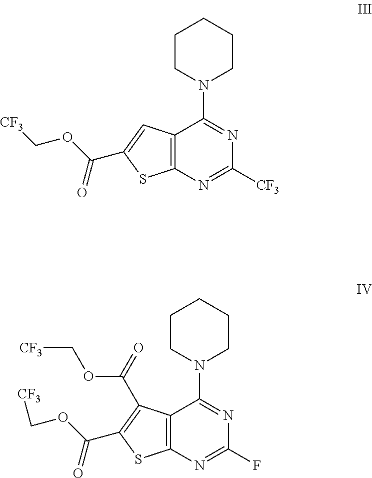 [SMILES: O=C(OCC(F)(F)F)c1cc2c(N3CCCCC3)nc(C(F)(F)F)nc2s1 ; O=C(OCC(F)(F)F)c1sc2nc(F)nc(N3CCCCC3)c2c1C(=O)OCC(F)(F)F]

4-12. (canceled)
